(19) United States
(12) Patent Application Publication
(10) Pub. No.: US 2026/0223889 A1
(43) Pub. Date: Aug. 6, 2026

(54) PROCESS FOR PREPARING GUANIDINOACETIC ACID COMPRISING GRANULATES

(71) Applicant: Evonik Operations GmbH, Essen (DE)

(72) Inventors: Wilfried BLUEMKE, Schoeneck (DE); Martin HEINING, Karlstein am Main (DE); Juliane MERZ, Dortmund (DE); Ansgar OELMANN, Gelnhausen (DE); Alexander MAYER, Planegg (DE)

(73) Assignee: Evonik Operations GmbH, Essen (DE)

(21) Appl. No.: 19/152,261

(22) PCT Filed: Jan. 29, 2024
(86) PCT No.: PCT/EP2024/052058

§ 371 Date: Jul. 30, 2025

(30) Foreign Application Priority Data
Feb. 6, 2023 (EP) 23155152

(51) Int. Cl. A23K 40/10; A23K 10/12; A23K 20/142; B01J 2/16; B01J 2/28; C12P 13/04

(52) U.S. Cl. CPC A23K 40/10; A23K 10/12; A23K 20/142; B01J 2/16; B01J 2/28; C12P 13/04

(57) ABSTRACT

The present application relates to a process for preparing guanidinoacetic acid comprising granulates, comprising the steps of a) providing a fermentation broth comprising guanidinoacetic acid and biomass, b) reducing the content of water in the fermentation broth provided in step a) to give a concentrated fermentation broth, and c) subjecting the concentrated fermentation broth of step b) to a wet granulation.

The present invention relates to a process for preparing guanidinoacetic acid comprising granulates, specific guanidinoacetic acid comprising granulates and a method for supplementation an animal diet with said guanidinoacetic acid comprising granulates.

Guanyl compounds, i.e., compounds with a guanidine group, are widely used as energy supplements, antidiabetic drugs, anti-inflammatory drugs, antihistamines, and hypotensive drugs. Many of these guanyl compounds are synthesized by transamidination reactions that transfer the amidino group of arginine to various amidino group acceptors, such as guanidinoacetate, 3-guanidinopropionic acid, 4-guanidinobutyric acid, 2-guanidinoethanol, hydroxy-guanidine, and homoarginine, among others. Biocatalytic transamidination reactions that use arginine substrates are usually reversible and are inhibited by the coproduct ornithine. Due to the inhibition by ornithine and the requirement for the expensive substrate arginine, the biosynthesis of some guanyl compound still remains a challenge.

Guanidinoacetic acid (GAA) that acts as a direct precursor of creatine, has recently attracted new interest as a nutritional additive due to its creatine-recovery effect and its high stability in aqueous solutions. In addition, it is also widely used in the pharmaceutical industry and as a feed additive in poultry farming. The European Feed Safety Authority (2009) concluded that GAA had no mutagenic or genotoxic properties and did not pose a risk to the environment. Given the wide utilization of GAA, there is a significant industrial demand for it.

In 1861, GAA was first prepared by chemically reacting cyanamide with glycine. Currently, GAA is mostly chemically synthesized by reacting glycine or sodium glycinate with guanylation agents, such as O-alkylisourea or cyanamide. The purification process of GAA is cumbersome due to the contamination of the final products with initial guanylation agents or toxic substances, such as iminodiacetic acid or methyliminodiacetic acid, and the production process is environmentally unfriendly. Therefore, biotechnical GAA production from renewable sources is highly desirable and holds promise to produce GAA.

GAA can be synthesized from arginine and glycine by arginine: glycine amidinotransferase (AGAT, EC: 2.1.4.1) in some vertebrates, however, only a minority of prokaryotes such as cyanobacteria can produce GAA in specialized metabolite synthesis. AGAT catalyzes the reversible transfer reaction of the amidino group from arginine (donor) to the amino group of glycine (acceptor) to produce GAA and ornithine. To produce one mole of GAA, one mole of arginine is required, and one mole of ornithine is produced.

The downstream process for a chemically produced guanidinoacetic acid comprises the steps of crystallization, mechanical dewatering of the thus obtained crystals comprising guanidinoacetic acid, e.g., GAA, with washing, drying of the crystals, granulating the dried crystals with a granulating (gluing) agent, and drying the granulates to give a free flowing and low dust generating product.

This rather complex downstream process can be simplified when guanidinoacetic acid, GAA, is produced in a fermentation process. In principle, the broth from said fermentation process can be directly fed to granulation.

For example, WO 2022/243116 A1 discloses a method for the fermentative production of guanidino acetic acid (GAA), comprising the steps of cultivating a suitable microorganism in a suitable medium under suitable conditions, and accumulating the GAA in the medium to form a GAA containing fermentation broth. This document also discloses the said method my further comprise the step of frying and/or granulating the GAA containing fermentation broth.

DE 1031366 A1 discloses that the granulation of feed additives, comprising amino acids and/or vitamins and optionally components of fermentation broths, is carried out in a circulating fluidized bed. The document also discloses granulated feed additives comprising 40-100 wt. % L-amino acids and up to 20 wt. % fermentation broth components and/or biomass formed during fermentation.

DE 102007034102 A1 discloses abrasion-resistant and free-flowing glycocyamine-containing moldings, in particular granules and extrudates, and to processes for their production. The moldings have a bulk density between 350 and 850 kg/m³, a grain size of 32 to 2750 μm and a glycocyamine content of 55 to 99.9% by weight, based on the total weight, and are particularly suitable as feed additives.

However, it is observed that such a fermentation broth tends to settle when not being agitated, and its granulation is inefficient. It is believed that these effects result from the low amount of dry matter that is typically contained in a fermentation broth, and the low viscosity. For example, a GAA fermentation broth has a dry matter content of less than 10 wt.-%, and the particle size distribution of the crystals contained therein is small, for example the GAA crystals in the fermentation broth have a particle size distribution of d10.3:4.62 μm; d50.3=10.50 μm; and d90.3=136.6 μm. In detail, it was observed that the long run times of the granulator due to the high content of water in the fermentation broth led to a low increase in dry matter inside the granulator. To make things even worse, the low increase in dry matter is superimposed by the abrasion between the particles, which results in a decrease in particle size from the initial d50.3 of 343 μm down to, for example, a d50.3 of 206 μm.

Accordingly, there was still a need for a process for preparing guanidinoacetic acid, GAA, comprising granulates, which overcomes the problems mentioned above. Such a process should allow the production of the guanidinoacetic acid, GAA, comprising granulates without the use of a binder.

It was found that the problems above are solved in that the content of water in the fermentation broth is reduced to give a concentrated fermentation broth, prior to being subject to a wet granulation.

One object of the present invention is therefore a process for preparing guanidinoacetic acid comprising granulates, comprising the steps of
a) providing a fermentation broth comprising guanidinoacetic acid and biomass,
b) reducing the content of water in the fermentation broth provided in step a) to give a concentrated fermentation broth, and
c) subjecting the concentrated fermentation broth of step b) to a wet granulation.

The process according to the present invention is not only simpler, and more cost effective but also more sustainable than the standard processes since it does not give any waste streams. Rather, all material coming from the fermentation process, apart from the water withdrawn in step b) and any water vapor in the wet granulation of step c), is contained in the final product. The product obtained from the process according to the present invention therefore comprises additional biomass, which is not only consumable for the animal but also provides an additional nutritional value. By comparison, the standard processes for preparing guanidinoacetic acid comprising granulates start from a chemically produced guanidinoacetic acid which involve further steps in the downstream process involving a crystallization, mechanical de-watering and washing, drying, mechanical granulation with the help of a granulation agent, followed by further drying.

In an embodiment of the process according to the present invention the fermentation broth is concentrated to a dry matter content of at least 10 wt.-% in step b).

Preferably, the fermentation broth is concentrated to a dry matter content of at least 15, 20, 25, 30 or 35 wt.-% in step b).

In a preferred embodiment of the process according to the present invention the fermentation broth is concentrated to a dry matter content of from 40 to 60 wt.-% in step b).

Preferably, the fermentation broth provided in step a) and/or obtained in step b) has a biomass content ranging from 1 to 10 wt.-%, or from 1 to 5 wt.-%, each based on the total weight of the fermentation broth or concentrated fermentation broth.

In the context of the present invention, the term wet granulation is used as known to the person skilled in the art and denotes the formation of granules by subjecting a liquid formulation containing suspended or dissolved solids to an agitating system, for example under the influence of an impeller (in a high-shear granulator), screws (in a twin screw granulator) or air (in a fluidized bed granulator). The agitation resulting in the system along with the wetting of the components within the formulation results in the aggregation of the primary powder particles to produce wet granules. The granulation liquid (fluid) contains a solvent or carrier material which must be volatile so that it can be removed by drying, and depending on the intended application, be non-toxic. Typical liquids used in wet granulation include water, ethanol, and isopropanol either alone or in combination, and the liquid solution can be either aqueous based or solvent based. Aqueous solutions have the advantage of being safer to deal with than other solvents. Since a fermentation broth is an aqueous system/formulation, it meets these advantages.

In principle, the wet granulation of the process according to the present invention is not subject to any limitation with respect to a specific wet granulation technique. Therefore, the wet granulation can be a high-shear granulation, a twin-screw granulation or a fluidized bed granulator. However, fluidized bed granulation has several advantages over other wet granulation techniques. These advantages are among others:
A fluidized bed granulator is a one-unit system, which makes fluidized-bed granulation a relatively simple process, and thus saves labor cost, transfer loss, and time.
This technique uses an air stream to evaporate liquids and thus avoids waste streams.
The heat transfer in a fluidized bed granulator is 2 to 6 times greater than that generated by tray dryer.
The process can be automated once parameters are optimized.
Drying occurs uniformly and the process prevents staining.

These aspects contribute to make the process according to the present invention simpler, and more cost effective.

In another embodiment of the process according to the present invention the wet granulation in step c) is a fluidized bed granulation.

The water and dissolved molecules (or solids) contained in the fermentation broth of step a) can form bonds between the particles already present in said fermentation broth that are strong enough to stick them together. The binding of the particles together with the use of liquid is a combination of capillary and clinging forces until more permanent solid bonding is established. However, once the thus obtained granulates are dried, they may fall apart. Therefore, water may not be strong enough to create and hold a bond. The fermentation broth used in the process according to the present invention contains biomass, which can hold or draw together the guanidinoacetic acid particles to form the granulates. Therefore, the biomass in the fermentation broth may already act as a granulation agent or binder. However, in cases, where the biomass does not provide this function at all or not to the extent as required, it is beneficial to add a granulation agent during the process according to the present invention.

In one embodiment of the process according to the present invention a granulation agent is added to the concentrated fermentation broth obtained in step b), prior to step c).

In principle, any binder or binding agent that holds or draws other materials together to form a cohesive whole mechanically, chemically, by adhesion or cohesion, can be used as granulation agent. However, a granulation agent for use in the preparation of guanidinoacetic acid comprising granulates, should not be toxic and should not have its own effect on an animal. In addition, any potential incompatibility with guanidinoacetic acid must be excluded. The use of an organic binders as granulation agent in the process according to the present invention offers the advantage that they are typically non-toxic and compatible with guanidinoacetic acid.

In a preferred embodiment of the process according to the present invention the granulation agent is an organic binder.

Organic binders, such as starch, cellulose ethers, cellulose esters, polyvinyl alcohols, and/or mixtures thereof, hold or draw the solid material together to form a cohesive whole mechanically, chemically, by adhesion or cohesion. In addition, they are also nontoxic and compatible with guanidinoacetic acid.

In another preferred embodiment of the process according to the present invention the granulation agent is a starch, a cellulose ether, a cellulose ester, a polyvinyl alcohol, and/or a mixture thereof.

It is beneficial to keep the amount of the granulation agent in the granulates to be prepared as low as possible. Thus, the concentration of guanidinoacetic acid and of any additional mass with further nutritional value is not unnecessarily decreased. In the process according to the present invention it only needs up to 5 wt.-% at the most, preferably from 1 to 5 wt.-% or from 2 to 4 wt.-% of the granulation agent, based on the mass of the dry binder on the dry matter in the fermentation broth.

In yet another embodiment of the process according to the present invention the amount of the granulation agent is 5 wt.-% at the most, based on the mass of the dry binder with respect to the dry matter in the fermentation broth.

Preferably, the amount of the granulation agent ranges from 1 to 5 wt.-% or from 2 to 4 wt.-%, based on the mass of the dry binder with respect to the dry matter in the fermentation broth.

Fluidized bed spray granulation can be defined as a particle-forming process by which a solid containing liquid is converted into non-dusty granular solids in one step by means of drying. Essentially, hot air is used to fluidize a bed of particles already formed (seed particles), which are either identical with or different from the dissolved component. In the context of the present invention the liquid to be processed is a solution or a suspension and is continuously sprayed, by means of an atomizing nozzle, either onto the bed from the space above the upper bed surface or into the bed.

The solvent is evaporated by the hot fluidizing, leaving behind the dissolved material on the surface of the seed particles. If the dissolved solids in the sprayed liquid and the particles in the fluidized bed consist of the same material, the process is called layering granulation. If a different solid is sprayed, the particles are coated, forming a distinctive layer on the seed particles. If the sprayed droplets hit the fluidized particles (seed particles) and do not rebound, they will spread and eventually dry on the particle surface, leaving behind the solids, which form a layer around the particle. Thus, layer-wise growth of the particles is achieved. The resulting particles are called granules or granulates. It is preferred that the process according to the present invention is a layering granulation. Hence, the seed particles provided in the fluidized fed granulator also comprise or consist of guanidinoacetic acid.

In a further embodiment of the process according to the present invention the wet granulation in step c) is an agglomeration granulation.

In the context of the present invention guanidinoacetic acid comprising seed particles having a d50.3 from 300 to 400 μm have been successfully used to prepare guanidinoacetic acid comprising particles having a particle size distribution d10.3 from 220 to 720 μm, d50.3 from 300 to 1100 μm, and d90.3 from 420 1700 μm. In particular, the seed particles have a particle size distribution of d10.3 from 150 to 250 μm, d50.3 from 300 to 400 μm, and d90.3 from 475 to 575 μm.

Preferably, seed particles having a d50.3 from 300 to 400 μm are used in step c) of the process according to the present invention.

The examples of the present invention show that granulates comprising guanidinoacetic acid and biomass and having a particle size distribution d10.3 from 220 to 720 μm, d50.3 from 300 to 1100 μm, and d90.3 from 420 to 1700 μm have improved or at least beneficial particle properties, like a good flowability, and/or improved particle qualities, like a high bulk density.

Another object of the present invention is therefore granulates comprising guanidinoacetic acid and biomass, wherein the granulates have the following particle size distribution d10.3 from 220 to 720 μm, d50.3 from 300 to 1100 μm, and d90.3 from 420 to 1700 μm.

In principle, the granulates according to the present invention are not subject to any limitation regarding the process for their preparation, provided that the process in question allows to give the granulates with the required particle size distribution. Nevertheless, it is preferred that said guanidinoacetic acid comprising granulates are obtained by the process according to the present invention. That is because the process according to the present invention, involving the wet granulation of step c), further improves product quality aspects like particle size or instant properties by gluing together the already existing solids to larger granulates. In addition to being simpler, more cost effective, and more sustainable, the process according to the present invention, involving the specific granulation step, further leads to improved product quality aspects of the guanidinoacetic acid comprising particles, like an increased particle size distribution having a particle size distribution d10.3 from 220 to 720 μm, d50.3 from 300 to 1100 μm, and d90.3 from 420 to 1700 μm, and a high bulk density, as well as improved particle properties, like a good flowability.

The examples of the present invention show that the use of a granulation agent, e.g., binder, is not mandatory for providing guanidinoacetic acid comprising granulates. Nevertheless, there may be cases, where the additional presence of a granulation agent, e.g., binder, is beneficial to hold the granulates together in particular under difficult or extreme conditions, for example, under pressure influence, such as mixing.

In an embodiment the granulates according to the present invention further comprise a granulation agent.

Preferably, said granulation agent is an organic binder, in particular a starch, a cellulose ether, a cellulose ester, a polyvinyl alcohol, and/or a mixture thereof.

In one embodiment the granulates according to the present invention have the following particle size distribution d10.3 from 300 to 720 μm, d50.3 from 450 to 1100 μm, and d90.3 from 700 to 1700 μm, preferably a particle size distribution d10.3 from 300 to 720 μm, d50.3 from 600 to 1100 μm, and d90.3 from 800 to 1700 μm.

Guanidinoacetic acid is widely used as energy supplement, antidiabetic drug, anti-inflammatory drug, antihistamine, and hypotensive drug.

A further object of the present invention is a method of supplementing an animal diet, wherein said diet is supplemented with guanidinoacetic acid comprising granulates according to the present invention and/or with the guanidinoacetic acid comprising granulates obtained by the process according to the present invention.

### EXAMPLES

### I. Example 1 According to the Invention

A fermentation broth with a biomass content of 45 g/kg was provided. The used fluidized bed granulator consisted of an electrical heater to heat the gas (air, nitrogen, CO₂ or combinations thereof) to an elevated temperature (180° C.), which then flowed through a distribution tray to fluidize the seed particles (d50.3:343 μm). A spray nozzle fed by a pump was used for atomizing the fermentation broth to fine droplets that dried on the seed particles and formed a solid layer on the surface or the seed particles or acted as a binder to combine seed particles to a larger agglomerate or combinations thereof. The fluidizing gas passed a filter before being released to the environment.

The broth with a dry matter of less than 10 wt.-%, containing biomass and crystals with a particle size distribution of d10.3:4.62 μm; d50.3=10.50 μm; and d90.3=136.6 μm tended to settle when not being agitated. This could lead to problems in the handling of the broth because of the settling of the particles and in the granulation because the low dry matter content makes the granulation inefficient. Specifically, the high content of water resulted in a low increase in dry matter inside the granulator, which led to long run times of the granulator. Because of the long run times, the abrasion between the particles was increased and led to a decrease in the particle size of the initial d50.3 value from 343 μm to for example 206 μm.

Therefore, the broth was evaporated in a vacuum evaporation at 50 to 100 mbara to a dry matter content of up to 50 wt.-%. Besides the advantage of less water in the broth, the increased viscosity also resulted in a more homogenous liquid because of the decreased precipitation velocity and thus decreased settling tendency of the suspended particles.

The following fluidized bed granulation was caried out with varying granulation agents, such as corn starch, CMC (carboxy methyl cellulose), and PVA (polyvinyl alcohol), in a range of from 2 to 4 wt.-% (mass dry binder on dry matter in broth). An increase in particle size distribution (PSD) could be achieved with every additive used and every concentration.

Since the granulation experiments with granulation agents led to an increase in particle size distribution, an addition granulation experiment without a granulation agent was also carried out. Surprisingly, a significant increase in particle size distribution was also seen when an evaporated fermentation broth was subjected to granulation with an additional granulation agent.

TABLE 1
Overview of the first run of granulation experiments
with and without granulation agent

| | Granulation agent | d10.3 | d50.3 | d90.3 |
|---|---|---|---|---|
| Ex. number | [wt.-%] | [μm] | [μm] | [μm] |
| Seed material | — | 204 | 343 | 525 |
| 1 | Corn starch, 3.03 | 426 | 650 | 1112 |
| 2 | CMC, 2.87 | 712 | 1067 | 1634 |
| 3 | PVA K90, 3.96 | 411 | 712 | 1097 |
| 4 | — | 327 | 508 | 751 |

### II. Example 2 According to the Invention

For verifying these results and for investigating the influence of the initial biomass concentration, a second run of experiments was carried out without any additional granulation agents or something similar. Here, a fermentation broth was used with 15 g biomass per kg broth that was evaporated to a dry matter content of about 50 wt.-%. In this run, the results of the preceding experiments could be repeated, in particular a significant increase in particle size distribution was found again. A significantly smaller starting material was used to highlight the effect of the growth in particle size distribution without reaching the limitations of the experimental equipment for fluidizing the material.

TABLE 2
Overview of the second run of granulation
experiments without granulation agent

| | d10.3 [μm] | d50.3 [μm] | d90.3 [μm] |
|---|---|---|---|
| Starting material | 6.21 | 17.14 | 76.88 |
| End material | 232.7 | 313.8 | 432.9 |

### III. Determination of the Flowability

The flowability of the particles was determined using 5 different funnels, each with a different orifice diameter: The first funnel had an orifice diameter of 2.5 mm, being the smallest orifice diameter. Product that passed this orifice was graded with a flowability value of 1. Next were the second to fourth funnels, having orifice diameters of 5 mm, 8 mm, and 12 mm. Product that passed the orifice of the second funnel was graded with a flowability value of 2, product that passed the orifice of the third funnel was graded with a flowability value of 3, and product that passed the orifice of the fourth funnel was graded with a flowability value of 4. The fifth and last funnel had an orifice diameter of 18 nm, and product that passed this orifice was graded with a flowability value of 5. Any product that did not pass the orifice of the fifth funnel was graded with a flowability value of 6. A flowability value of 5 or more indicates that the product in question may not be suitable for automated, high output feed mills. Table 3 below summarizes he results of the determination of the flowability.

### IV. Determination of the Angle of Repose

Flow characteristics of powdered substances are determined by measuring the height of the constituted cone. For the experiment, a sieve with a mesh size, which the particles should pass unobstructed, was fixed in a distance of 60 mm above the top of a metal cylinder. The substance to be evaluated was gently rubbed through the sieve until a geometrically constant cone was formed on top of the metal cylinder. The angle of response was calculated by means of the formula:

$$Angle of repose [ ° ] = {tan}^{- 1} \frac{H}{r}$$

with
H=height of the cone in mm
r=radius of the metal cylinder in mm

### V. Determination of Bulk Density (ρ_{b}) and Tamped Apparent Density (ρₜ)

For the bulk density, the mass of a defined volume of powdered or granulated substance weas determined. The tamped apparent density indicated the changed volume after tamping of the mass obtained at the bulk density measurement. Here, a standardized test equipment was used consisting of a metal tripod, a funnel with a defined tulip-shaped outlet, and a rotary slide. The test equipment was placed on top of a 250 mL glass cylinder. The test substance was filled into the metal funnel, and then the rotary slide was opened to let the test substance run out evenly until 20 ml of the test substance was filled into the cylinder. The bulk density was then calculated by means of the following formula:

$$bulk density [ \frac{kg}{{m}^{3}} ] = \frac{{m}_{test sample} [ g ]}{{V}_{initial} [ mL ]} \times 1000$$

$$with$$

$${V}_{initial} = 250 mL .$$

The tamped apparent density was determined according to DIN 53194 using a tamping device, e.g., JEL ST 2, Fa. Engelsmann AG, Ludwigshafen. The tamped apparent density was calculated by means of the following formula:

$$tamped apparent density [ \frac{kg}{{m}^{3}} ] = \frac{{m}_{test sample} [ g ]}{{V}_{tampled} [ mL ]} \times 1000$$

### VI. Determination of the Carr Index

The Carr index is an indicator of the compressibility of a powder and is calculated by means of the formula:

$$Carr factor = 100 \times ( 1 - ( \frac{{ρ}_{b}}{{ρ}_{t}} ) )$$

with
ρ_{b}=bulk density
ρₜ=tamped apparent density

The results of the determination of flowability, angle of repose, bulk density, tamped apparent density, and Carr index are summarized in Table 3.

TABLE 3
Handling properties of the granulates from examples 1 and 2 commercially available
products GuanAmino ® (from Evonik) or CreAmino ® (from Alzchem Group).

| | | Example 1: | Example 2: | | |
|---|---|---|---|---|---|
| | | biomass | biomass | | |
| | | granulate w/o | granulate w/o | | |
| Handling | | binder, 45 g/kg | binder, 15 g/kg | | |
| properties | Unit | biomass | biomass | GuanAmino ® | CreAmino ® |
| flowability | grade | 2 | 2 | 2 | 2 |
| angle of repose | [°] | 31 | 30.6 | 36.8 | 31.5 |
| bulk density | [kg/m³] | 565 | 657 | 645 | 649 |
| tamped | [kg/m³] | 637 | 744 | 732 | 733 |
| apparent | | | | | |
| density | | | | | |
| Carr index | — | 11.3 | 11.6 | 11.8 | 11.4 |

A comparison of the data in Table 3 shows that the products obtained by the process according to the present invention, examples 1 and 2, are of the same or at least comparable quality as the comparative material, i.e., the commercially available products GuanAmino® (from Evonik) or CreAmino® (from Alzchem Group). However, the comparative material was prepared in a chemical process, and the thus obtained material was further processed in a highly complex process. By comparison, the product of examples 1 and 2 was obtained from the significantly less complex process according to the present invention. The effect of a higher water uptake is probably due to the hygroscopic biomass in the product but can be dealt with.

### VII. Determination of the Particle Size Distribution

The particle size distribution (PSD) is measured by laser diffraction method. This method uses the effect that a particle will scatter light at a certain angle depending on the particle's size. Smaller particles at wide angles, larger particles at small angles. Hence, a certain pattern of light can be detected depending on the overall PSD of the measured sample.

Here, the PSD was measured with a laser diffraction particle size analyzer LA 950 (Horiba). The measured sample was suspended in iso-propanol. Before measuring, the sample was treated with ultrasound for 60 s or 120 s. Alternatively, the sample was measured without ultrasound. It was found that the sample measured with no ultrasonic treatment delivered higher values, whereas the 60 s and 120 s sample delivered comparable results in the range of the accuracy of the measurement. Therefore, the values measured after 60 s ultrasonic treatment are mentioned here.

### VIII. Summary

In a comprehensive experimental investigation, it was shown that it is possible to produce guanidinoacetic acid, GAA, comprising granulates from a guanidinoacetic acid comprising fermentation broth by evaporating and granulating, in particular by fluidized bed granulation. The standard process starts from a chemically produced product that is subjected to a complex downstream process involving crystallization, mechanical dewatering and washing, drying, mechanical granulation with the help of a granulation agent and again drying. By comparison, the process according to the present invention is much simpler. In detail, the process according to the present invention is simpler, more cost effective and more sustainable than the standard process. It is a further benefit of the process according to the present invention that it does not give waste streams except from the water from evaporation and the water vapor from the wet granulation, e.g., fluidized bed granulation, since all material coming from the fermentation process is contained in the final product. Further, all remainder from the fermentation have a potential nutritional value and can be consumed by animals. Finally, the granulates obtained from the process according to the present invention are of the same or at least similar quality as the products from standard processes.

1. A process for preparing guanidinoacetic acid comprising granulates, the process comprising:
a) providing a fermentation broth comprising guanidinoacetic acid and biomass,
b) reducing a content of water in the fermentation broth provided in a) to obtain a concentrated fermentation broth, and
c) subjecting the concentrated fermentation broth of b) to a wet granulation.

2. The process according to claim 1, wherein the fermentation broth is concentrated to a dry matter content of at least 10 wt. % in b).

3. The process according to claim 1, wherein the fermentation broth is concentrated to a dry matter content of from 40 to 60 wt. % in b).

4. The process according to claim 1, wherein the wet granulation in c) is a fluidized bed granulation.

5. The process according to claim 1, wherein a granulation agent is added to the concentrated fermentation broth obtained in b), prior to c).

6. The process according to claim 5, wherein the granulation agent is an organic binder.

7. The process according to claim 5, wherein the granulation agent is at least one selected form the group consisting of a starch, a cellulose ether, a cellulose ester, a polyvinyl alcohol, and a mixture thereof.

8. The process according to claim 5, wherein an amount of the granulation agent is 5 wt. % or less, based on a mass of a dry binder with respect to a dry matter in the fermentation broth.

9. The process according to claim 1, wherein the wet granulation in c) is an agglomeration granulation.

10. Granulates comprising guanidinoacetic acid and biomass, wherein the granulates have a particle size distribution d10.3 from 220 to 720 μm, d50.3 from 300 to 1100 μm, and d90.3 from 420 to 1700 μm.

11. The granulates according to claim 10, wherein the granulates further comprise a granulation agent.

12. The granulates according to claim 10, wherein the granulates have the particle size distribution d10.3 from 300 to 720 μm, d50.3 from 450 to 1100 μm, and d90.3 from 700 to 1700 μm.

13. A method of supplementing an animal diet, the method comprising:
supplementing the animal diet with guanidinoacetic acid comprising the granulates of claim 10.

14. A method of supplementing an animal diet, the method comprising:
supplementing the animal diet with guanidinoacetic acid comprising granulates obtained by the process of claim 1.
